(19) United States
(12) Patent Application Publication
(10) Pub. No.: US 2001/0056163 A1
(43) Pub. Date: Dec. 27, 2001

(54) Macromonomer compounds, their preparation from diene compounds and their use

(76) Inventors: Dominique Charmot, Le Pre-Saint-Gervais (FR); Philippe Chaumont, Lyon (FR); Daniel Colombani, Strasbourg (FR)

Correspondence Address: RHODIA INC., CN-7500, 259 Prospect Plains Road, CRANBURY, NJ, 08512, US

(21) Appl. No.: 09/896,297

(22) Filed: Jun. 29, 2001

Related U.S. Application Data
(63) Continuation of application No. 09/101,858, filed on Oct. 26, 1998.

(30) Foreign Application Priority Data
Jan. 17, 1996 (FR) 96/00466

(51) Int. Cl. C07D 327/02

(52) U.S. Cl. 526/317.1; 526/328; 526/335; 526/341; 549/90; 549/549

(57) ABSTRACT

The present invention relates to macromonomer compounds having the formula (I) where R1 is hydrogen, halogen, and optionally substituted alkyl, aryl, alkylaryl or arylalkyl radical; R2 and R3, which can be similar or different, are hydrogen, halogen, alkyl, aryl, alkylaryl, arylalkyl or alkoxy radical; R4 is an optionally substituted alkyl, aryl, alkylaryl or arylalkyl radical, or —XR5, with R5 being hydrogen, and optionally substituted alkyl, aryl, alkylaryl or aryalkyl; X is oxygen or sulphur; A corresponds to a unit issued from at least one ethylenically unsaturated monomer; and n is comprised between 1 and 10000. The invention also relates to dienic compounds having the formula (II): H2C═CR1—CH═CH—C(R2)(R3)—X2—R6 wherein R1 R2, R3 and X have the same meaning as above and R6 represents R5 or a group —COR7 or —COOR7 with R7 being alkyl, aryl, alkylaryl or arylalkyl.

The subject of the present invention is macromonomer compounds in which one of the ends exhibits a ring containing three atoms, including a heteroatom, and diene compounds exhibiting a terminal functional group of peroxy or disulphide type.

The present invention likewise relates to a process for the preparation of the abovementioned macromonomer compounds from the said diene compounds by radical polymerization.

Finally, the invention relates to the use of the said macromonomer compounds for the preparation of copolymers.

Patent Application WO 91/06535 describes transfer agents of H₂C═C(R¹)(CX₂—O—O—R²) type, in which formula the R¹ radical represents a hydrogen atom, a chlorine atom, an alkyl group or a group capable of activating the double bond and R² represents a hydrogen atom, optionally substituted alkyl, alkenyl or aryl groups or a —CO—Z group in which Z corresponds to an R group of the optionally substituted alkyl, alkenyl or aryl type or an —OR group in which R has the same meaning as above. The R¹ radical is preferably a group which activates the double bond, of the aryl, cyano or alternatively ester type.

The polymers obtained by radical polymerization in the presence of the abovementioned transfer agents with unsaturated monomers exhibit a terminal epoxy group. However, this terminal epoxy group is disubstituted, due to the presence of the abovementioned R¹ radical, which is other than a hydrogen atom.

The process described in the abovementioned patent application does not make it possible to obtain all the possible types of epoxy groups. Indeed, the monosubstituted rings cannot be synthesized because the corresponding transfer agents (R¹=H) are not sufficiently reactive.

The present invention therefore relates to the preparation of macromonomer compounds comprising, at one of their ends, a ring containing three atoms, one of which is an oxygen or sulphur atom.

Such macromonomer compounds exhibit the unexpected advantage of being more reactive in polymerization than their homologues described in the prior art. In particular, these novel macromonomer compounds are more reactive with monomers, polymers or copolymers comprising at least one functional group of carboxyl, carboxylate, amine, alcohol and/or thiol type.

Thus, the subject of the present invention is macromonomer compounds of following formula (I):

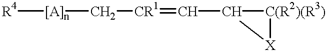

in which formula:

R¹ represents:

a hydrogen atom, a halogen atom or a carboxyl, alkoxycarbonyl, acyloxy, carbamoyl or cyano group,

an alkyl, aryl, arylalkyl or alkylaryl radical, these radicals optionally being substituted by at least one carboxyl, alkoxycarbonyl, acyloxy, carbamoyl or cyano group,

R² and R³, which are identical or different, represent a hydrogen atom, a halogen atom or an alkyl, aryl, alkylaryl, arylalkyl or alkoxy radical,

R⁴ represents:

an alkyl radical or an aryl, alkylaryl or arylalkyl radical, these radicals optionally being substituted by at least one carboxyl, alkoxycarbonyl, acyloxy, carbamoyl or cyano group, or

a radical —XR⁵, with R⁵ representing a hydrogen atom or an alkyl, aryl, alkylaryl or arylalkyl radical, these radicals optionally being substituted by at least one carboxyl, alkoxycarbonyl, acyloxy, carbamoyl or cyano group,

X represents an oxygen atom or a sulphur atom,

A corresponds to a unit resulting from at least one monomer containing ethylenic unsaturation, and

n is between 1 and 10,000.

Another subject of the present invention relates to diene compounds of following formula (II): H₂C═CR¹—CH═CH—C(R²) (R³)—X₂—R⁶, in which formula the R¹, R², R³ and X radicals have the same meaning as above and the radical R⁶ represents:

an alkyl, aryl, alkylaryl or arylalkyl radical, these radicals optionally being substituted by at least one carboxyl, alkoxycarbonyl, acyloxy, carbamoyl or cyano group,

a —CO—R⁷ or —COOR⁷ group with R⁷ representing an alkyl, aryl, alkylaryl or arylalkyl radical,

a hydrogen atom, and, when R² and R³ represent a methyl group, R⁶ does not represent a hydrogen atom.

The present invention also relates to the use of the compounds of formula (II) for the preparation of the macromonomer compounds of formula (I).

Finally, the invention relates to the use of the macromonomer compounds of formula (I) for the preparation of copolymers.

However, other advantages and characteristics of the present invention will become more clearly apparent on reading the following description and examples.

For greater clarity, the reactive diene compounds of formula (II) used for the synthesis of the macromonomer compounds (I) according to the invention will first of all be described.

As has just been shown, the diene compounds are compounds of following formula (II): H₂C═CR¹—CH═CH—C(R²)(R³)—X₂—R⁶, in which formula R¹, R², R³, R⁶ and X have been defined above.

According to a specific embodiment of the invention, the R¹ radical represents a hydrogen atom or a carboxyl, alkoxycarbonyl, acyloxy, carbamoyl or cyano group. The R¹ radical preferably represents a hydrogen atom.

According to a specific form of the invention, the R² and R³ radicals, which are identical or different, represent a hydrogen atom or a linear or branched C₁-C₆ alkyl radical.

Furthermore, the R⁵ radical more particularly represents a hydrogen atom or an alkyl, aryl, alkylaryl or arylalkyl radical, these radicals optionally being substituted by at least one carboxyl, alkoxycarbonyl, acyloxy, carbamoyl or cyano group. According to an advantageous embodiment of the present invention, R⁵ more particularly represents an alkyl, aryl, alkylaryl or arylalkyl radical.

According to a preferred embodiment of the invention, R⁶ more particularly represents an alkyl, aryl, alkylaryl or arylalkyl radical, these radicals optionally being substituted by at least one carboxyl, alkoxycarbonyl, acyloxy, carbamoyl or cyano group.

According to a preferred embodiment of the present invention, the diene compounds of formula (II) are such that X represents an oxygen atom.

Such compounds can be synthesized from their halogenated derivatives, which are brought into contact with a derivative of peroxide or disulphide type.

This reaction is commonly carried out in solvent medium. Mention may be made, among suitable solvents, of cyclic ethers, such as tetrahydrofuran, or any compound capable of dissolving the intermediate cations of the reaction, such as hydroperoxide or disulphide salts, or the halogenated derivative present. Mention may be made, for example, of crown ethers or alternatively polyethylene oxides with a mass of 400.

The reaction is generally carried out at a temperature such that the degradation reactions by thermolysis of the reactants and the products obtained are negligible. By way of information, the reaction temperature is less than room temperature and more particularly less than 10° C.

The macromonomer compounds of formula (I), which are the subject of the present invention, exhibit R¹, R², R³, R⁴ and X radicals as defined above.

According to a specific embodiment of the invention, the R¹ radical represents a hydrogen atom or a carboxyl, alkoxycarbonyl, acyloxy, carbamoyl or cyano group. The R¹ radical preferably represents a hydrogen atom.

According to a specific form of the invention, the R² and R³ radicals, which are identical or different, represent a hydrogen atom or a linear or branched C₁-C₆ alkyl radical.

The R⁵ radical more particularly represents a hydrogen atom or an alkyl, aryl, alkylaryl or arylalkyl radical, these radicals optionally being substituted by at least one carboxyl, alkoxycarbonyl, acyloxy, carbamoyl or cyano group.

According to an advantageous embodiment of the present invention, R⁵ more particularly represents an alkyl, aryl, alkylaryl or arylalkyl radical.

According to one embodiment of the invention, R⁶ more particularly represents an alkyl, aryl, alkylaryl or arylalkyl radical, these radicals optionally being substituted by at least one carboxyl, alkoxycarbonyl, acyloxy, carbamoyl or cyano group.

According to a preferred embodiment of the present invention, the macromonomer compounds of formula (I) are such that X represents an oxygen atom.

The macromonomer compounds (I) according to the invention additionally comprise, in their structure, n groups A, with A corresponding to a unit resulting from a monomer containing ethylenic unsaturation and n of between 1 and 10,000. Preferably, n is between 1 and 5000.

The target, as monomer containing ethylenic unsaturation, is more specifically according to the invention the monomers chosen from styrene or its derivatives, butadiene, (meth)acrylic esters and vinyl nitriles.

(Meth)acrylic esters denotes esters of acrylic acid and of methacrylic acid with C₁-C₁₂ alcohols, preferably C₁-C₈ alcohols. Mention may be made, among the compounds of this type, without intending to be limited thereto, of methyl acrylate, ethyl acrylate, propyl acrylate, n-butyl acrylate, isobutyl acrylate, 2-ethylhexyl acrylate, methyl methacrylate, ethyl methacrylate, n-butyl methacrylate or isobutyl methacrylate.

The vinyl nitrites include more particularly those having from 3 to 12 carbon atoms, such as, in particular, acrylonitrile and methacrylonitrile.

It should be noted that styrene can be replaced, in all or in part, by derivatives, such as α-methylstyrene or vinyltoluene.

The other ethylenically unsaturated monomers which can be used, alone or as mixtures, or which can be copolymerized with the above monomers are, in particular:

vinyl esters of carboxylic acid, such as vinyl acetate, vinyl versatate or vinyl propionate,

unsaturated ethylenic mono- and dicarboxylic acids, such as acrylic acid, methacrylic acid, itaconic acid, maleic acid or fumaric acid, and monoalkyl esters of dicarboxylic acids of the type mentioned with alkanols preferably having 1 to 4 carbon atoms, and their N-substituted derivatives,

amides of unsaturated carboxylic acids, such as acrylamide, methacrylamide, N-methylolacrylamide or N-methylolmethacrylamide,

ethylenic monomers containing a sulphonic acid group and its alkali metal or ammonium salts, for example vinylsulphonic acid, vinylbenzenesulphonic acid, α-acrylamidomethylpropanesulphonic acid or 2-sulphoethylene methacrylate,

unsaturated ethylenic monomers containing a secondary, tertiary or quaternary amino group or a heterocyclic group containing nitrogen, such as, for example, vinylpyridines, vinylimidazole or aminoalkyl (meth)acrylates and aminoalkyl(meth)acrylamides, such as dimethylaminoethyl acrylate or methacrylate, di-tert-butylaminoethyl acrylate or methacrylate, or dimethylaminomethylacrylamide or dimethylaminomethylmethacrylamide. It is likewise possible to use zwitterionic monomers, such as, for example, sulphopropyl(dimethyl)aminopropyl acrylate.

As has been shown previously, the macromonomer compounds of formula (I) are obtained from the diene compounds of formula (II). In this case, the formula of the diene compounds of formula (II) includes, moreover, R⁶ equal to a hydrogen atom when R² and R³ both represent a methyl radical.

More particularly, for the preparation of the said monomers (I), a radical polymerization is carried out with at least one abovementioned monomer containing ethylenic unsaturation and at least one diene compound of formula (II).

The polymerization is carried out in a way known per se, in solution, in bulk or in aqueous emulsion, when the monomer or monomers used are immiscible with water, in the presence of at least one radical initiator and of at least one diene compound of formula (II).

Any type of free radical initiator usual in radical polymerization may be suitable.

Examples of initiators comprise hydroperoxides, such as hydrogen peroxide or diisopropylbenzene hydroperoxide, sodium, potassium or ammonium persulphates and azo initiators, such as azobis(isobutyronitrile) or 4-4′-azobis(4-cyanovaleric acid).

These initiators can be used in combination with a reducing agent, such as, for example, bisulphite. The amount is generally between 0.05 and 2% by weight with respect to the amount of the monomers.

The amount of diene compound of formula (II) depends on the molecular weight desired for the macromonomer of formula (I). By way of information, this amount is generally between 0.05 and 10%, preferably between 0.1 and 3%, by weight with respect to the total weight of the monomers.

The diene compound can be introduced into the reaction mixture, either all at the beginning of the reaction, or continuously in solution in the main monomers, or alternatively partly at the beginning and partly continuously.

When the solubility of the product in the monomers is low, it can be introduced in the form of a suspension simultaneously with the said monomers.

The polymerization temperature is a function of the nature of the initiator and the person skilled in the art is able, with his own knowledge of the field, to determine the appropriate temperature. More particularly, the reaction is carried out at a temperature such that the degradation reactions by thermolysis of the reactants and the products obtained are negligible. By way of information, the reaction is carried out at temperatures of less than 100° C.

Advantageously, the conversion of the diene compound to macromonomers is not limited and it is possible to achieve quantitative conversions of the latter without degrading the macromonomer obtained.

In the case where the polymerization is carried out in aqueous emulsion, the stabilization of the particles is provided, if necessary, by any known colloidal stabilization system, such as anionic, cationic, amphoteric and non-ionic emulsifiers.

The polymerization can be carried out continuously, non-continuously or semi-continuously with introduction of a portion of the monomers continuously and can be of the “seeded” or “incremental” type, according to any alternative form known for the production of particles of homogeneous and heterogeneous structure.

The compounds of general formulae (I) made it possible to synthesize copolymers exhibiting specific structures, such as copolymers of grafted or comb structure.

Thus, according to a first possibility, grafted or comb copolymers are obtained (i) by reacting the macromonomer compounds (I) with at least one monomer containing ethylenic unsaturation exhibiting at least one carboxyl, carboxylate, amine, alcohol or thiol functional group and then (ii) by copolymerizing by a radical route the macromonomers (I), thus modified, with at least one other monomer containing ethylenic unsaturation.

The radical polymerization takes place in the presence, generally, of an effective amount of a free-radical initiator.

The lists shown above relating to the monomers and initiators remain valid and will not be repeated here.

The relative amounts of monomers and of macromonomer compounds used in the second stage are variable and depend on the percentage of grafting of the grafted copolymer which it is desired to obtain. The person skilled in the art knows how to adapt these relative amounts as a function of the result which is sought.

However, it is recommended to use from 0.1 to 60%, preferably from 1 to 30%, by weight of macromonomer with respect to the weight of the grafted copolymer finally obtained.

According to another alternative form of the invention, the macromonomer compounds (I) according to the invention can be used to prepare, simply and efficiently, copolymers of comb or grafted structure.

Thus, at least one polymer or copolymer containing functional groups which are capable of reacting with the ring containing three atoms comprising sulphur, and more particularly oxygen, is reacted.

Mention may be made, as functional groups of this type, of polymers or copolymers exhibiting at least one carboxyl, carboxylate, amine, alcohol and/or thiol functional group.

Such a reaction is generally carried out in the presence of a catalyst, such as, for example, a quaternary ammonium salt in the case of addition of a group to the oxirane ring of the macromonomer.

According to a third alternative form, the macromonomer compounds according to the invention, more particularly those for which X represents oxygen, can result in comb copolymers, a polymerization being carried out by ring opening. In this case, the reaction can be catalysed by strong bases, for example sodium methoxide, or by Lewis acids, for example BF₃.(C₂H₅)₂O.

The copolymers obtained by the process of the invention have numerous applications.

They may, for example, be used as compatabilizing agents in polymer mixtures, agents for adhesion to substrates, dispersing agents, or agents for resistance to ageing and for improving gloss in polymer coatings.

They can additionally be used to chemically modify natural gums (for example: guar, xanthan, and the like), as associative polymers, sequestering polymers, surface-active polymers or thermoplastic elastomer polymers.

The examples and tests below illustrate the invention without limiting the scope thereof.

### EXAMPLE 1

Preparation of cumyl 2,4-pentadienyl Peroxide (CPDP)

A. Preparation of 5-bromo-1,3-pentadiene

A solution comprising 40 g of PBr₃ in 75 ml of anhydrous diethyl ether is added dropwise to a mixture, cooled to a temperature of −10° C., of 25 g of 3-hydroxy-1,4-pentadiene in 75 ml of dry diethyl ether. The combined mixture is maintained at room temperature with stirring for 12 hours.

The reaction mixture is then cooled to −20° and then 200 ml of distilled water are subsequently added.

The product is extracted with diethyl ether and then dried over MgSO₄. The solvent is then evaporated under reduced pressure and 31 g of 5-bromo-1,3-pentadiene are obtained.

B. Preparation of cumyl 2,4-pentadienyl peroxide (CPDP)

7.26 g of potassium hydroxide (85%) are added little by little with stirring to a solution, cooled to −5° C., comprising 14.7 g of 5-bromo-1,3-pentadiene obtained above, 17.8 g of cumene hydroperoxide (94% Fluka) and 1 g of polyoxyethylene (POE-di-OH 400, Hoechst) in 150 ml of tetrahydrofuran.

The reaction temperature is maintained below 0° C. The reaction mixture is then brought to room temperature and filtered.

The filtrate is concentrated by evaporating under vacuum, then mixed with 10 ml of water and then extracted with 3 times 30 ml of heptane.

The organic phases are dried over MgSO₄ and then distilled under vacuum to remove the solvent.

The product is purified by liquid solid chromatography on a silica gel column, by gel permeation.

12 g of a product comprising 92% of the E isomer and 8% of the Z isomer are obtained.

### EXAMPLE 2

Preparation of the Macromonomer Compounds from Cumyl 2,4-pentadienyl Peroxide.

Mother solutions containing monomers and initiator are prepared from 40 ml of distilled monomer, the initiator (2,2-azobis(isobutylonitrile) being incorporated in this volume in the following proportions:

| | | Methyl | Butyl | Methyl |
|---|---|---|---|---|
| Monomer | Styrene | methacrylate | acrylate | acrylate |
| Initiator | 40 | 40 | 8 | 8 |
| (mg) | | | | |

5 ml of the mother solutions are introduced into test tubes and variable amounts of the cumyl 2,4-pentadienyl peroxide prepared in Example 1 are added, these amounts being collated in the table below.

The tubes are deaerated and sealed under vacuum.

The reactions are carried out at 60° C. for a time such that the conversion remains below 10%. It is thus 1 hour for the styrene and the methyl methacrylate monomers and 10 minutes for the butyl acrylate and methyl acrylate monomers.

The reactions are then stopped by addition of hydroquinone and then cooling by immersion of the tubes in a bath containing ice and isopropanol.

The resulting polymers are precipitated from heptane or methanol and then dried.

The conversions of the monomers are measured by gravimetry, from the weights of the polymers and of the initial amount of macromonomer.

The molecular weights are determined by gel permeation chromatography. The results are expressed as absolute molecular masses, except for poly(methyl acrylate), the molecular weight of which is expressed as poly(butyl acrylate) equivalent.

The relationship between the (number) degree of polymerization DPₙ and the molar ratio of cumyl 2-4-pentadienyl peroxide and monomer involved in the polymerization makes it possible to measure a transfer constant Ctr by using the Mayo relationship [F. R. Mayo, J. Am. Chem. Soc., 65, 2324 (1943)].

This constant Ctr makes it possible to measure the ability of cumyl 2,4-pentadienyl peroxide to react in the presence of the monomer under radical polymerization conditions to produce macromonomers containing epoxy ends. The reactivity of the diene compounds (II) improves as Ctr increases.

| Tests | Monomer (M) | 10⁴ [CPDP]/[M] | DPₙ | Ctr |
|---|---|---|---|---|
| 1 | MMA | 0 | 4556 | 3.1 |
| 2 | MMA | 0.56 | 2441 | |
| 3 | MMA | 2.80 | 995 | |
| 4 | MMA | 5.59 | 552 | |
| 5 | MMA | 11.2 | 268 | |
| 6 | MMA | 28.0 | 110 | |
| 7 | St | 0 | 1731 | 1.1 |
| 8 | St | 0.56 | 1442 | |
| 9 | St | 2.81 | 1106 | |
| 10 | St | 5.61 | 837 | |
| 11 | St | 11.2 | 548 | |
| 12 | St | 28.1 | 268 | |
| 13 | MA | 0 | 3415 | 3.1 |
| 14 | NA | 0.59 | 3387 | |
| 15 | NA | 2.96 | 945 | |
| 16 | NA | 5.92 | 476 | |
| 17 | NA | 11.8 | 177 | |
| 18 | NA | 29.6 | 62 | |
| 19 | BA | 0 | 12,320 | 4.7 |
| 20 | BA | 0.59 | 2327 | |
| 21 | BA | 2.96 | 554 | |
| 22 | BA | 5.92 | 260 | |
| 23 | BA | 11.8 | 162 | |
| 24 | BA | 29.6 | 63 | |

MMA: methyl methacrylate

St: styrene

MA: methyl acrylate

BA: butyl acrylate

[CPDP]/[M]: ratio of the concentration of the cumyl 2,4-pentadienyl peroxide to that of the monomer used.

DPn: number-average degree of polymerization, equivalent to n in the definition given of the macromonomer (I)

### EXAMPLE 3

This example shows that the reaction of the diene compound CPDP with a monomer results in the formation of a macromonomer possessing a terminal epoxy group.

With this aim, a low-mass polymer (Mn=1680 g/mol) is prepared from methyl methacrylate according to the same method as described in Example 2, a high amount [concentration of 0.8 mol/l] of the macromonomer obtained in Example 1 being used.

The polymer obtained is precipitated and washed in methanol, in order to remove all trace of remaining reactant.

The ¹H NMR spectrum of the polymer obtained exhibits the characteristics according to the table below. This NMR spectrum shows the existence of a vinyloxirane group at the end of the chain.

| Shift (ppm) | Interpretation |
|---|---|
| 5.8-4.8 | presence of two ethylenic |
| | protons (2H) |
| 3.6 | presence of three protons |
| | of a methoxycarbonyl |
| | group |
| 1.6 | presence of three protons |
| | of the methyls of the |
| | cumyl radical |
| 3.7-3.2 | presence of a proton of |
| | the epoxide fragment |
| 3.0-2.5 | presence of two protons |
| | of the epoxide fragment |

### EXAMPLE 4

Preparation of Tert-butyl Pentadiene Peroxide (TBPDP)

14 g (250 mmol) of KOH are added portionwise to a solution, cooled to −5° C., of 22 g (150 mol) of 1-bromo-2,4-pentadiene, 23 g (230 mmol) of t-butyl hydroperoxide and 2 ml of poly-oxoethylene 400 in 60 ml of THF and then the mixture is stirred for 24 h while being allowed to return to room temperature. The reaction mixture is filtered and the filtrate is concentrated under reduced pressure, then mixed with 50 ml of water and extracted three times with heptane. After drying over NgSO₄ and evaporating the solvents, the crude reaction product is quickly chromatographed on a silica column (eluent: 3/17 ether/pentane). 15.5 g of a pale-yellow oil are obtained (100 mmol, 57%).

### EXAMPLE 5

Preparation of the Polystyrene Macromonomer Compounds Resulting from the TBPDP of Example 4.

750 mg (4.8 mmol) of TBPDP, 10.4 g (100 mmol) of styrene and 8 mg (0.049 mmol) of AIBN are introduced into a tube with a pinched neck. The tube is sealed under reduced pressure after degassing and then heated at 80° C. for 6 h 30. The mixture is precipitated from 200 ml of methanol, the polymers collected dried under vacuum and then dissolved in 20 ml of THF and again precipitated from 200 ml of methanol. After drying under vacuum, 2.50 g of polymers are recovered (24% conversion).

The ¹H NMR spectrum of the polymer obtained exhibits the same characteristics as those of the polymer of Example 3, which show the existence of a vinyloxirane group at the end of the chain.

### EXAMPLE 6

Preparation of 6-cumylperoxy-6-methoxy-2,4-hexadiene (CPMH)

The acetal 3,3-dimethoxy-1-propene is obtained by reaction of 2,4-hexadienal (1.92 g, 0.0020 mol) with trimethyl orthoformate (0.021 mol, 2.23 g) in the presence of para-toluenesulphonic acid (25 mg, 0.001 mol). Cumyl hydroperoxide (94%, 3.40 g, 0.021 mol) is then added dropwise at room temperature to the acetyl obtained. The methanol released is removed as it is formed under water pump vacuum (10 mm Hg) and the peroxyketal is purified by chromatography on silica gel (50 g) (2.95 g, 60%); R_{f}=0.47, 90/10 heptane/Et₂O.

### EXAMPLE 7

A. Preparation of the poly(methyl methacrylate) macromonomer compounds resulting from CPMH. The initiator 2,2′-azobisisobutyronitrile (40 g, 2.44×10⁻⁴ mol at 60° C.) is dissolved in distilled MMA (40 ml). A fixed volume (5 ml) of this solution is placed in clean and dry Pyrex tubes. A solution of 6-cumylperoxy-6-methoxy-2,4-hexadiene (5×10⁻⁴ mol) in MKA (10 ml) is also prepared and a variable amount is added to the different phials. The polymerization is carried out at seven different concentrations of transfer agent of 0 to 2.5×10⁻² mol/l. The reaction mixtures are degassed three times and then the tubes are sealed under reduced pressure (0.01 mm Hg). The polymerizations are carried out at a temperature of 60° C. for a time of 60 minutes.

The transfer constant Ctr measured is 0.09.

B. Preparation of the Polystyrene Macromonomer Compounds Resulting from CPMH.

The preparation is similar to that of Example 7A, the methyl methacrylate is replaced by styrene.

The transfer constant Ctr measured is 0.14.

### EXAMPLE 8

Preparation of Polystyrene Macromonomer Compounds Resulting from ethyl 2-(tert-butylperoxymethyl)acrylate (ETBPA).

ETBPA is disclosed by the general formula I in the document WO 91/06535, with R¹=CO₂Et, X=H and R²=tert-butyl.

A. Preparation of ETBPA

A solution of 85% KOH in 50 ml of THF is added portionwise to a solution, cooled to −10° C., of 7.51 g (38.9 mmol) of ethyl (2-bromomethyl)acrylate, 4.35 g (41.6 mmol) of tert-butyl hydroperoxide (titrated at 86% w/w) and 0.8 ml of poly-oxoethylene 400 di-OR. The reaction lasted 18 h.

After treatment, the crude reaction product is chromatographed on silica (eluent: ether/pentane, 10%). 0.93 g of a pale-yellow oil is obtained (5 mmol, 44%).

B. Preparation of the Macromonomer

610 mg (3.01 mmol) of ETBPA, 10.4 g (100 mmol) of styrene and 8 mg (0.049 mmol) of AIBN are introduced into a tube with a pinched neck. The tube is sealed under reduced pressure after degassing and then heated at 80° C. for 6 h 30. The mixture is precipitated from 200 ml of methanol, the polymers collected dried under vacuum and then dissolved in 20 ml of THF and again precipitated from 200 ml of methanol. After drying under vacuum, 3.60 g of polymers are recovered (35% conversion).

The ¹H NMR spectrum reveals a peak at 3.2 ppm characteristic of the 2 protons of the oxirane unit at the end of the chain.

Test: Reaction of the Macromonomers Containing Epoxy Ends with Methacrylic Acid.

This comparative test is targeted at demonstrating the greater reactivity, with respect to ethylenic monomers containing a carboxyl functional group, of the macromonomers containing a vinyloxirane end in agreement with the invention (Example 5), relative to the macromonomers according to the prior art prepared in Example 8.

A. A solution of 100 mg of macromonomer prepared in Example 5, 210 mg of methacrylic acid and 35 mg of ethanol in 1 ml of THF is heated at 60° C. for 1 hour. After addition of 2 ml of THF, the polymers are precipitated from 50 ml of methanol and are recovered after filtration on sintered glass and drying under vacuum.

B. A solution of 100 mg of macromonomer prepared in Example 8, 210 mg of methacrylic acid and 35 mg of ethanol in 1 ml of THF is heated at 60° C. for 1 hour. After addition of 2 ml of THF, the polymers are precipitated from 50 ml of methanol and are recovered after filtration on sintered glass and drying under vacuum.

The degree of reaction is measured from the integration areas of the ¹H NMR peaks corresponding to the protons of the epoxy units (3.2-2.6 ppm) and to the protons of the ethylenic bond groups of the methacrylate residue.

| | Yield of the reaction |
|---|---|
| Macromonomer of Example 5 | 70% |
| Macromonomer resulting from | 0% |
| Example 8 | |

1. Macromonomer compounds of following formula (I): 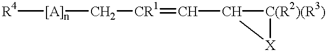
in which formula:
R¹ represents a hydrogen atom, a halogen atom, a carboxyl, alkoxycarbonyl, acyloxy, carbamoyl or cyano group or an alkyl, aryl, arylalkyl or alkylaryl radical, these radicals optionally being substituted by at least one carboxyl, alkoxycarbonyl, acyloxy, carbamoyl or cyano group,
R² and R³, which are identical or different, represent a hydrogen atom, a halogen atom or an alkyl, aryl, alkylaryl, arylalkyl or alkoxy radical,
R⁴ represents an alkyl radical or an aryl, alkylaryl or arylalkyl radical, these radicals optionally being substituted by at least one carboxyl, alkoxycarbonyl, acyloxy, carbamoyl or cyano group, or —XR⁵, with R⁵ representing a hydrogen atom or an alkyl, aryl, alkylaryl or arylalkyl radical, these radicals optionally being substituted by at least one carboxyl, alkoxycarbonyl, acyloxy, carbamoyl or cyano group,
X represents an oxygen atom or a sulphur atom,
A corresponds to a unit resulting from at least one monomer containing ethylenic unsaturation, and
n is between 1 and 10,000.

2. Macromonomer compounds according to any one of the preceding claims, characterized in that the monomer containing ethylenic unsaturation is chosen from styrene or its derivatives, butadiene, methacrylic acid and its esters containing from 1 to 8 carbon atoms, acrylic acid and its esters containing from 1 to 8 carbon atoms, vinyl esters, vinyl nitriles or their mixtures.

3. Diene compounds of following formula (II):
H₂C═CR¹—CH═CH—C(R²)(R³)—X₂—R⁶
in which formula:
R¹ represents a hydrogen atom, a halogen atom, a carboxyl, alkoxycarbonyl, acyloxy, carbamoyl or cyano group or an alkyl, aryl, arylalkyl or alkylaryl radical, these radicals optionally being substituted by at least one carboxyl, alkoxycarbonyl, acyloxy, carbamoyl or cyano group,
R² and R³, which are identical or different, represent a hydrogen atom, a halogen atom or an alkyl, aryl, alkylaryl, arylalkyl or alkoxy radical,
R⁵ represents a hydrogen atom or an alkyl, aryl, alkylaryl or arylalkyl radical, these radicals optionally being substituted by at least one carboxyl, alkoxycarbonyl, acyloxy, carbamoyl or cyano group,
R⁶ represents:
an alkyl, aryl, alkylaryl or arylalkyl radical, these radicals optionally being substituted by at least one carboxyl, alkoxycarbonyl, acyloxy, carbamoyl or cyano group,
a —CO—R⁷ or —COOR⁷ group with R⁷ representing an alkyl, aryl, alkylaryl or arylalkyl radical, or
a hydrogen atom,
and, when R² and R³ represent a methyl group, R⁶ does not represent a hydrogen atom,
X represents an oxygen atom or a sulphur atom.

4. Compounds according to any one of the preceding claims, characterized in that the R¹ radical represents a hydrogen atom or a carboxyl, alkoxycarbonyl, acyloxy, carbamoyl or cyano group.

5. Compounds according to any one of the preceding claims, characterized in that the R² and R³ radicals, which are identical or different, represent a hydrogen atom or a linear or branched C₁-C₆ alkyl radical.

6. Compounds according to any one of the preceding claims, characterized in that the R⁵ radical represents a hydrogen atom or an alkyl, aryl, alkylaryl or arylalkyl radical, these radicals optionally being substituted by at least one carboxyl, alkoxycarbonyl, acyloxy, carbamoyl or cyano group.

7. Compounds according to the preceding claim, characterized in that the R⁶ radical represents an alkyl, aryl, alkylaryl or arylalkyl radical, these radicals optionally being substituted by at least one carboxyl, alkoxycarbonyl, acyloxy, carbamoyl or cyano group.

8. Compounds according to any one of the preceding claims, characterized in that X represents an oxygen atom.

9. Use of a diene compound of formula (II) according to any one of claims 3 to 8 for preparing macromonomer compounds of formula (I) according to any one of claims 1 to 2 and 4 to 8.

10. Process for the preparation of macromonomer compounds (I) according to any one of claims 1 to 2 and 4 to 8, characterized in that a radical polymerization reaction is carried out with:
at least one monomer containing ethylenic unsaturation and
at least one diene compound of formula (II) according to any one of claims 3 to 8.

11. Process for the preparation of macromonomer compounds (I) according to any one of claims 1 to 2 and 4 to 8, characterized in that a radical polymerization reaction is carried out with:
at least one monomer containing ethylenic unsaturation, and
at least one diene compound of formula (II) in which:
R¹ represents a hydrogen atom, a halogen atom, a carboxyl, alkoxycarbonyl, acyloxy, carbamoyl or cyano group or an alkyl, aryl, arylalkyl or alkylaryl radical, these radicals optionally being substituted by at least one carboxyl, alkoxycarbonyl, acyloxy, carbamoyl or cyano group,
R² and R³, which are identical or different, represent a hydrogen atom, a halogen atom or an alkyl, aryl, alkylaryl, arylalkyl or alkoxy radical,
R⁵ represents a hydrogen atom or an alkyl, aryl, alkylaryl or arylalkyl radical, these radicals optionally being substituted by at least one carboxyl, alkoxycarbonyl, acyloxy, carbamoyl or cyano group,
R⁶ represents:
an alkyl, aryl, alkylaryl or arylalkyl radical, these radicals optionally being substituted by at least one carboxyl, alkoxycarbonyl, acyloxy, carbamoyl or cyano group,
a —CO—R⁷ or —COOR⁷ group with R⁷ representing an alkyl, aryl, alkylaryl or arylalkyl radical, or
a hydrogen atom,
X represents an oxygen atom or a sulphur atom.

12. Preparation process according to claim 10 or 11, characterized in that the amount of diene compound of formula (II) is between 0.05 and 10% by weight with respect to the total weight of the monomers containing ethylenic unsaturation.

13. Process for the preparation of copolymers of comb or grafted structure from the macromonomer compounds according to any one of claims 1 to 2 and 4 to 8.
